(19) United States
(12) Patent Application Publication
(10) Pub. No.: US 2004/0191779 A1
(43) Pub. Date: Sep. 30, 2004

(54) Statistical analysis of regulatory factor binding sites of differentially expressed genes

(76) Inventors: Jie Zhang, Campbell, CA (US); Hsiu-Ying Wei, San Jose, CA (US); Leslie Margaret McEvoy, Mountain View, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/401,830

(22) Filed: Mar. 28, 2003

(51) Int. Cl. C12Q 1/68; G06F 19/00; G01N 33/48; G01N 33/50

(52) U.S. Cl. 435/6; 702/20

(57) ABSTRACT

The invention concerns the statistical analysis of regulatory factor binding sites of differentially expressed genes. More particularly, the invention concerns methods for identifying and characterizing regulatory factor, e.g. transcription factor binding sites in differentially expressed genes in order to develop therapeutic strategies for the treatment of diseases which are accompanied by differential gene expression, or to study biological processes.

### CROSS-REFERENCE TO RELATED APPLICATIONS

This application is related to U.S. Ser. No. ______, filed ______, entitled “Genomic Profiling of Regulatory Factor Binding Sites”, and identified as Attorney Docket No. 39753-0001, the entire disclosure of which is hereby expressly incorporated by reference.

### BACKGROUND OF THE INVENTION

1. Field of the Invention

The present invention concerns the statistical analysis of regulatory factor binding sites of differentially expressed genes. More particularly, the invention concerns methods for identifying and characterizing regulatory factor, e.g. transcription factor binding sites in differentially expressed genes in order to develop therapeutic strategies for the treatment of diseases which are accompanied by differential gene expression.

2. Description of the Related Art

One of the main approaches to identify novel therapeutic targets is the study of differential gene expression, typically comparing normal and diseased biological samples, or biological samples representative of different stages of a particular disease or pathologic condition. In general, methods used to study differential gene expression can be based on hybridization analysis and/or sequencing of polynucleotides. The most commonly used methods known in the art for the quantification of differential gene expression in a sample include northern blotting and in situ hybridization (Parker & Barnes, Methods in Molecular Biology 106:247-283 (1999)); polymerase chain reaction (PCR) (Weis et al., Trends in Genetics 8:263-264 (1992)), such as quantitative real-time PCR, and microarray analysis. Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

Differential gene expression studies have been conducted on a variety of human tissues and biological samples representing a verity of biological processes, such as various cancers, neuronal diseases, developmental disorders, aging processes, infectious diseases, and the like.

### SUMMARY OF THE INVENTION

The present invention is based on the recognition that the large number of differentially expressed genes identified in a biological sample, which may be, but need not be, representative of various diseases, disease states and other abnormalities, is the result of changes in the transcription functioning of a handful of regulatory factors, such as transcription factors (TF).

In one aspect, the present invention concerns a method for statistical analysis of differentially expressed genes, comprising:

(a) obtaining a set of differentially expressed genes

(b) screening genomic sequences including the regulatory regions of the differentially expressed genes for the presence of regulatory factor binding sites; and

(c) identifying at least one regulatory factor binding site enriched within the set of differentially expressed genes relative to a genome-wide or tissue-wide background.

The set of differentially expressed genes can be obtained from results of differential gene or protein expression studies, and thus can, for example, be generated by microarray, RT-PCR, or proteomics approaches.

In step (c) enrichment may, for example, be determined by comparing the frequencies or probabilities of the occurrence of the regulatory binding site or binding sites identified in step (c) within the gene set.

In a particular embodiment, the set of differentially expressed genes may be part of a gene expression profile characteristic of a disease, disorder, or biological process. All diseases, disorders and biological processes associated with gene transcription are included, such as, without limitation, tumor, oncological diseases, neurological diseases, cardiovascular diseases, renal diseases, infectious diseases, digestive diseases, metabolic diseases, inflammatory diseases, autoimmune diseases, dermatological diseases, and diseases associated with trauma or abnormal skeletal development. Metabolic diseases specifically include, without limitation, diabetes, and diseases of lipid, carbohydrate and calcium metabolism. Dermatological diseases specifically include, without limitation, diseases requiring wound healing.

In a further specific embodiment, the disease is cancer, which can, for example, be breast cancer, renal cancer, leukemia, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, and brain cancer.

In another embodiment, the disorder is a developmental disorder.

In yet another embodiment, the biological process represented by the differentially expressed gene set is associated with aging.

In a further embodiment, the gene set consists of genes that show at least about two-fold , or at least about four-fold, or at least about ten-fold differential expression relative to control.

In a still further embodiment, the regulatory factor binding site is identified within a 5′ upstream core promoter region, a 5′ upstream enhancer region, an intron region, and/or a 3′ regulatory region.

In another embodiment, the regulatory factor binding site is a transcription factor binding site. Without limitation, and merely by way of illustration, the transcription factor can be selected from the group consisting of c-Fos, c-Jun, AP-1, Elk, ATF, c-Ets-1, c-Rel, CRF, CTF, GATA-1, POU1F1, NF-κB, POU2F1, POU2F2, p53, Pax-3, Sp1, TCF, TAR, TFEB, TCF-1, TFIIF, E2F-1, E2F-2, E2F-3, E2F-4, HIF-1, HIF-1α, HOXA1, HOXA5, Sp3, Sp4, TCF-4, APC, and STAT5A.

In a specific embodiment, the transcription factor is E2F-1, E2F-2, E2F-3, NF-κB, Elk, AP-1, c-Fos, or c-Jun.

Typically, a large number of differentially expressed genes is analyzed. Thus, the analysis may extend to at least about 100 differentially expressed genes, or at least about 500 differentially expressed genes.

In a further aspect, the invention concerns method for designing a treatment strategy based upon the identification of the enriched regulatory factor binding site(s) by the foregoing method.

In a specific embodiment, the enriched regulatory factor binding site is a transcription factor binding site binding to at least one transcription factor.

In a further embodiment, a consensus binding site is identified based on the enriched transcription factor binding site.

The treatment strategy may, for example, rely on the design of a double-stranded oligonucleotide decoy, which competes with said enriched binding site for binding to the corresponding transcription factor, or on an anti-sense oligonucleotide designed to bind to the mRNA of enriched transcription factor.

In a different aspect, the invention concerns a method of designing a consensus regulatory factor binding site, comprising identifying a regulatory factor binding site enriched within a set of differentially expressed genes, relative to a genome-wide or tissue-wide control, and designing a consensus regulatory factor binding site consisting essentially of nucleotides shared by the regulatory factor binding sites enriched within the set of differentially expressed genes.

In yet another aspect, the invention concerns a method of analyzing the enrichment of a regulatory factor binding site in a biological sample comprising a set of differentially expressed genes, comprising comparing the frequency or probability of the occurrence of the regulatory binding site within the gene set with the frequency or probability of its occurrence in a reference sample. The statistical analysis is preferably performed by using a hypergeometric distribution model.

### BRIEF DESCRIPTION OF DRAWINGS

FIG. 1 shows the frequencies of TF binding sites between G1 and S phase differentially expressed genes and whole genome background.

FIG. 2 is a graphical representation of the number of microarray-related publications between 1995 and 2002.

### DETAILED DESCRIPTION OF THE PREFERRED EMBODIMENT

A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

For purposes of the present invention, the following terms are defined below.

The term “regulatory factor,” is used in the broadest sense, and includes any factor that is capable of affecting the mRNA transcription process of genes. Specifically included within this term are transcription factors

The terms “gene regulatory sequence,” “cis-regulatory element,” “cis-acting regulatory element,” “cis-regulatory sequence,” and “cis-acting regulatory sequence” are used interchangeably, and refer to any regulatory sequence that controls gene expression, including, without limitation, 5′ regulatory regions and 3′-regulatory regions, such as, promoters, enhancers, silencers, transcription termination signals, and splicing signals; intron regions, and intergenic regions, and sequences that regulate translation. Specifically included are DNA recognition sequences with which transcription factors associate (also referred to as transcription factor binding sites).

The term “transcription factor binding site” refers to short consensus genomic sequences that locate immediately before the transcription start sites (TSS) of genes. A transcription regulatory region can contain several binding sites, and can therefore be bound by several transcription factors.

“Trans-factors” are proteins that bind to cis-regulatory sequences.

“Transcription factors” are proteins that bind to DNA near the transcription initiation site of a gene, and either assist or inhibit RNA polymerase in initiation and maintenance of transcription.

“DNA binding domain” is a region within a transcription factor that recognizes specific bases in a target gene near the transcription initiation site.

The “transcription starting site (TSS)” is the position where a gene's mRNA starts to be transcribed from DNA by RNA polymerase II

The term “transcription factor decoy” or “decoy” is used herein to refer to short double-stranded oligonucleotides that specifically bind target transcription factors, thereby preventing the transcription factors from initiating the transcription of their target genes.

The term “microarray” refers to an ordered arrangement of hybridizable array elements, preferably polynucleotide probes, on a substrate.

The term “polynucleotide,” when used in singular or plural, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The terms “differentially expressed gene,” “differential gene expression” and their synonyms, which are used interchangeably, refer to a gene whose expression is activated to a higher or lower level in a sample obtained from a subject suffering from a disease, relative to its expression in a normal or control (reference) sample. The terms also include genes whose expression is activated to a higher or lower level at different stages of the same disease. A differentially expressed gene may be either activated or inhibited at the nucleic acid level or protein level, or may be subject to alternative splicing to result in a different polypeptide product. Such differences may, for example, be evidenced by a change in mRNA levels, surface expression, secretion or other partitioning of a polypeptide. Differential gene expression may include a comparison of expression between two or more genes or their gene products, or a comparison of the ratios of the expression between two or more genes or their gene products, or even a comparison of two differently processed products of the same gene, which differ between normal subjects and subjects suffering from a disease, or between various stages of the same disease. Differential expression includes both quantitative, as well as qualitative, differences in the temporal or cellular expression pattern in a gene or its expression products among, for example, normal and diseased cells, or among cells which have undergone different disease events or disease stages. For the purpose of this invention, “differential gene expression” is considered to be “significant” when there is at least an about two-fold, preferably at least about four-fold, more preferably at least about six-fold, most preferably at least about ten-fold difference between the expression of a given gene in normal and diseased subjects, or in various stages of disease development in a diseased subject.

A “set” of differentially expressed genes includes sufficient number of genes for statistical analysis. In general, the set will include at least about 20, or at least about 50, or at least about 100, or at least about 200, or at least about 500, or at least about 1000 genes.

The term “treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy.

The term “tumor,” as used herein, refers to all neoplastic cell growth and proliferation, whether malignant or benign, and all pre-cancerous and cancerous cells and tissues.

The terms “cancer” and “cancerous” refer to or describe the physiological condition in mammals that is typically characterized by unregulated cell growth. Examples of cancer include but are not limited to, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

B. Detailed Description

The practice of the present invention will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2^{nd} edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4^{th} edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

The present invention is based on the systematic comparison of the regulatory regions of genes identified as being differentially expressed in a particular disease, disease state, or abnormality. In particular, the present invention is based on the recognition that a common link among the numerous differentially expressed genes is change in the transcription processes of a handful of regulatory, e.g. transcription, factors.

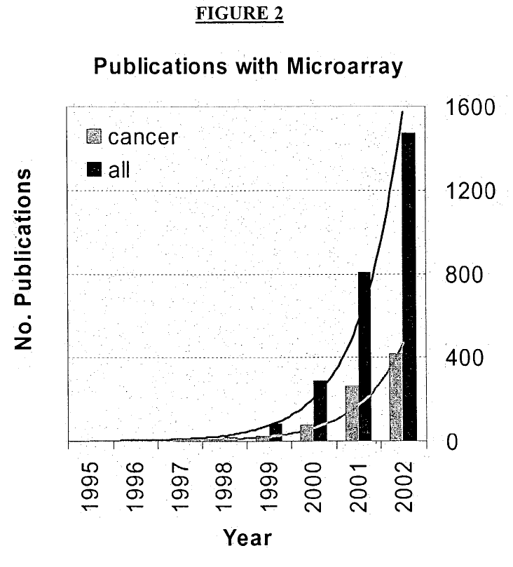

As noted before, researchers have a variety of techniques at their disposal to study differential gene expression. Although the most frequently used approaches are microarray and RT-PCR, other techniques, such as Northern blotting, RNase protection assays, differential plaque hybridization, subtractive hybridization, serial analysis of gene expression (SAGE; Velculescu et al., Science 270:484-487 (1995); and Velculescu et al., Cell 88:243-51 (1997)), rapid analysis of gene expression (RAGE; Wang et al., Nucleic Acids Research, 27:4609-18, (1999)), and massively parallel signature sequencing (MPSS; Brenner et al., Nature Biotechnology 18:630-634 (2000)), are equally suitable for the study of differential gene expression. More and more studies have been conducted about the differential gene expression. FIG. 2 gives an outline about the publications of microarray technology based all biomedical researches or cancer specific researches.

In the microarray method, polynucleotide sequences of interest (including cDNAs and oligonucleotides) are plated, or arrayed, on a microchip substrate. The arrayed sequences are then hybridized with specific DNA probes from cells or tissues of interest. In a specific embodiment of the microarray technique, PCR amplified inserts of cDNA clones are applied to a substrate in a dense array, typically including at least about 10,000 nucleotide sequences. The immobilized microarrayed genes are suitable for hybridization under stringent conditions. Fluorescently labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously, thereby providing differential gene expression data. Microarray analysis can be performed by commercially available equipment, following manufacturer's protocols, such as by using the Affymetrix GenChip technology, or Agilent's microarray technology.

RT-PCR can also be used to compare mRNA levels in different sample populations, such as in normal and diseased (e.g. tumor) tissues to characterize patterns of gene expression, to discriminate between closely related mRNAs, and to analyze RNA structure.

The first step is the isolation of mRNA from a target sample. As RNA cannot serve as a template for PCR, the first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

A more recent variation of the RT-PCR technique is the real time quantitative PCR, which measures PCR product accumulation through a dual-labeled fluorigenic probe (i.e., TaqMan® probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

Differential gene expression can also be studied at the protein level, using proteomics techniques. The proteome is the totality of the proteins present in a sample (e.g. tissue, organism, or cell culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D PAGE); (2) identification of the individual proteins recovered from the gel, e.g. mass spectrometry and/or N-terminal sequencing, and (3) analysis of the data using bioinformatics. Proteomics methods are valuable supplements to other methods of gene expression profiling, and can be used, alone or in combination with other methods, to study differential gene expression. For further details see, e.g. Proteomics in Practice: A Laboratory Manual of Proteome Analysis, R. Westermeier et al., eds., John Wiley & Sons, 2002.

Typically, gene expression studies identify hundreds to a few thousands of differentially expressed genes in the test samples, relative to normal samples. For example, studies in normal biological processes, such as HeLa cell cycles, and abnormal biological phenotype, such as rotavirus infected tissue, have shown that at least about 500 genes exhibit significant changes relative to their normal counterparts. Most of the gene expression data have been deposited into public and commercial databases, such as Stanford Micoarray Database (SMD), Yale Microarray Database, ArrayExpress at the European Bioinformatics Institute IEBI). These, and other publicly available gene expression databases are listed in Table 1 below.

TABLE 1

| Name of database | Description |
|---|---|
| ArrayExpress | A repository for microarray based gene |
| | expression data maintained by European |
| | Bioinformatics Institute. |
| ChipDB | A searchable database of gene expression. |
| ExpressDB | A relational database containing yeast and E. |
| | coli RNA expression data. |
| Gene Expression Atlas | A database for gene expression profile from |
| | 91 normal human and mouse samples across |
| | a diverse array of tissues, organs, and cell |
| | lines. |
| Gene Expression Database (GDX) | A database of Mouse Genome Informatics |
| | at the Jackson laboratory. |
| Gene Expression Omnibus | A database in NCBI for supporting the |
| | public use and disseminating of gene |
| | expression data. |
| GeneX | National Center for Genome Resource's |
| | imitative to provide an Internet-available |
| | repository of gene expression data. |
| Human Gene Expression Index (HuGE | Aims to provide a comprehensive database |
| Index) | to understand the expression of human |
| | genes in normal human tissues. |
| M-CHiPS (Multi-Conditional Hybridization | A data warehousing concept and focuses on |
| Intensity Processing System) | providing a structure suitable for statistical |
| | analysis of a microarray database's entire |
| | components including the experiment |
| | annotations. |
| READ (RIKEN cDNA Expression Array | A database maintained by RIKEN (The |
| Database) | institute of Physical and Chemical Research), |
| | Japan. |
| RNA Abundance Database (RAD) | RNA Abundance Database (RAD) is a public |
| | gene expression database designed to hold |
| | data from array-based and non-array-based |
| | (SAGE) experiments. The ultimate goal is to |
| | allow comparative analysis of experiments |
| | performed by different laboratories using |
| | different platforms and investigating different |
| | biological systems. |
| Saccharomyces Genome Database | A gene expression database of |
| (SGD): Expression Connection | Saccharomyces genome at Stanford |
| | University; provides simultaneous search of |
| | the results of several microarray studies for |
| | gene expression data for a given gene or |
| | ORF. |
| Stanford Microarray Database (SMD) | Stores raw and normalized data from |
| | microarray experiments, as well as their |
| | corresponding image files. In addition, SMD |
| | provides interfaces for data retrieval, analysis |
| | and visualization. Data is released to the |
| | public at the researcher's discretion or upon |
| | publication. |
| Yale Microarray Database | |
| Yeast Microarray Global Viewer | A database for yeast gene expression data |
| | maintained by Laboratoire de genetique |
| | moleculaire, Ecole Normale Superieure. |
| 3D-Gene Expression Database | Preliminary structure for a database of 3D- |
| | visualization of developmental gene |
| | expression. |
| BODYMAP | A databank of gene expression information |
| | of human and mouse genes, created by |
| | random sequencing of clones in 3′-directed |
| | cDNA libraries. |
| Gene Resource Locator | The goal is to map millions of ESTs to the |
| | human genome for the study of the exonintron |
| | structures of genes, the alternative |
| | splicing of pre-mRNAs, the promoter regions |
| | of full-length-enriched cDNA sequences, and |
| | the gene-expression patterns associated with |
| | ESTs. |
| RNA Abundance Database (RAD) | A public gene expression database designed |
| | to hold data from array-based and non-array- |
| | based (SAGE) experiments. The ultimate |
| | goal is to allow comparative analysis of |
| | experiments performed by different |
| | laboratories using different platforms and |
| | investigating different biological systems. |
| TissueInfo | An online database which determines the |
| | tissue expression profile of a sequence by |
| | comparing the given sequence against the |
| | EST database. Each EST comes from a |
| | library derived from a specific tissue type. |

Despite extensive research in this field and the large volume of accumulated data, in view of the complexity of gene expression, differential gene expression data are difficult to interpret.

It has been well accepted that it is very unlikely that each of the numerous differentially expressed genes has mutations or some other defects. On the contrary, it is possible that the large number of differentially expressed genes is the result of changes in a few key phenomena or mechanisms, which can affect simultaneously the expression levels of many genes. The present invention is based on the recognition that the large number of differentially expressed genes in various diseases, disease states or other abnormalities results from changes in a few regulatory factors, such as transcription factors (TF).

Transcription factors (TFs) are a class of proteins that control and initialize the process of transcribing genetic information coded by DNA into mRNA. All currently known TFs are classified into five different subfamilies, named after their functional domains, namely the Basic Domains, Zinc-coordinating DNA binding domain, Helix-turn-helix domains, beta-Scaffold Factors with Minor Groove Contacts, and Other Transcription Factors. Usually, at least a few transcription factors are required to form a transcriptional complex that binds to the regulatory regions of genes and, as a result, controls and initializes the mRNA transcription machinery. These binding processes are mediated by the DNA binding domains of TF proteins. It is known that only some of the transcription factors are capable of binding directly to DNA, while others are required to form the functional transcription machinery, without the requirement of direct binding to the regulatory regions of the target genes.

At the present time, there are more than 4000 known TF's, about 2000 of which are from mammalian species. Exemplary TFs, without limitation, include c-Fos, c-Jun, AP-1, ATF, c-Ets-1, c-Rel, CRF, CTF, GATA-1, POU1F1, NF-κB, POU2F1, POU2F2, p53, Pax-3, Sp1, TCF, TAR, TFEB, TCF-1, TFIIF, E2F-1, E2F-2, E2F-3, E2F-4, HIF-1, HIF-1α, HOXA1, HOXA5, Sp3, Sp4, TCF-4, APC, and STAT5A.

Of the mammalian TFs, only several hundred have been shown to have the ability to bind directly to the regulatory regions (cis-regulatory binding sites) of the target genes, and only a few hundred TF binding sites have been characterized up to date. The TF binding sites of genes are short stretches of DNA sequences located in the regulatory region of the genes. These sites are specific for different DNA binding TFs, and usually are about 6 to about 16 bases in length. It is known that within a given binding site there are bases at certain positions that are absolutely required for binding by the corresponding TF, while others can tolerate some base-change variations. For further details see, for example, Davidson, E. H., Genomic Regulatory Systems: development and evolution, ISBN 0-12-205351-6, Academic Press, 2001. and, for example, Michael Carey, Stephen T. Smale, Transcriptional Regulation in Eukaryotes, ISBN 0-87969-537-4, Cold Spring Harbor Laboratory Press, 2000.

There are several transcription factor related databases, which are listed in Table 2 below.

TABLE 2

| Database | TF | Sites | Address |
|---|---|---|---|
| TRANSFAC | factors | sites | http://transfac.gbf.de/ |
| | | | TRANSFAC/index.html |
| TRRD | factors | sites | http://wwwmgs.bionet.nsc.ru/ |
| | | | mgs/gnw/trrd |
| TFD | factors | sites | http://kisec.cmb.ki.se/kisac/ |
| | | | databases/tfd.html |
| COMPEL | compository | sites | http://compel.bbionet.nsc.ru/ |
| EPD | N/A | promoters | http://www.epd.isb-sib.ch/ |
| IMD | factors | sites | http://bimas.dcrt.nih.giv/molbio/ |
| | | | matrixs/ |

Of the listed databases TRANSFAC collects the most in terms of number of TF binding sites, and is updated and cited frequently (Heinemeyer et al., 1998, Heinemeyer et al., 1999, Karas et al., 1997, Knuppel et al., 1994, Matys et al., 2003, Wingender et al., 1996, Wingender et al., 1997, Wingender et al., 1997, Wingender et al., 2000., Wingender et al., 2001). The usage of TF binding sites for protein-pathway evaluation has been recently reported (Krull et al, 2003).

In the broadest sense, the present invention provides, for the first time, a method for the comparative analysis of regulatory regions of a large number of genes in order to identify common regulatory mechanisms and/or consensus regulatory factor binding sites shared by such genes. Accordingly, the present invention provides new insight into so far undiscovered relationships between such genes, and enables the identification of significant regulatory factors from the large amount of gene expression data available at the present time or to be generated in the future.

The idea underlying the present invention is that if one can identify certain consensus regulatory factor binding sites, such as, for example, TF binding sites, shared by most of the differentially expressed genes identified in various diseases, diseases states or abnormalities. If the certain regulatory factor, e.g. TF binding sites are found enriched among such differentially expressed genes relative to their tissue-wide or genome-wide existences, the identified binding sites very likely play a major role in the resultant differential expression and, in turn, could be responsible for the disease or abnormalities, such as the final cell-fate change seen in cancer or tumor.

In one particular aspect, the present invention provides a novel approach for comparative analysis of regulatory regions of differentially expressed genes in order to identify consensus regulatory regions enriched within such genes, which can then be used to identify one or more regulatory factors that play a role in the regulation of their expression.

In another aspect, the present invention provides a method for identifying regulatory factors, such as transcription factors (TFs), providing a link among the large number of genes differentially expressed in a disease, disease state or abnormality, by a systematic comparison of their regulatory regions.

As a result of their involvement in an essential regulatory mechanism associated with a disease process, the shared regulatory factor binding sites and the corresponding regulatory factors are valuable therapeutic-development targets. For example, by altering the TFs identified, for example, by antisense oligonucleotide approach (to bind the mRNA of the TF and in turn to alter the corresponding protein expression) or by changing the transcription effects of such TFs, e.g. by using the transcription decoy method (to competitively bind to corresponding TFs), new approaches can be developed for the treatment (including prevention) of a variety of diseases, disorders, and abnormalities, or for interfering with certain detrimental or undesired biological processes, such as aging. In a more generic sense, the present invention provides a valuable tool for biomedical studies and research efforts in general, and provides a unique tool for understanding such processes. In general, the information provided by the present invention can be utilized for a variety of different purposes and applications including but not limited to, biomedical research, pre-clinical development, drug screening applications, target discovering and target validation, building genome- or tissue-wide connections between regulatory profiles of different genes, understanding the genome or tissue background of various known regulatory factors, understanding the genome or tissue background of various known transcription factors, and the like.

Accordingly, the present invention is directed to a method for the statistical analysis of the regulatory factor (e.g. TF) binding sites of differentially expressed genes. In a particular aspect, the present invention provides new therapeutic targets by identifying regulatory, e.g. transcription factors that have been responsible for the differential expressions of a large number of genes found in a biological sample representative of a disease, disorder, or a particular biological process.

In a particular embodiment, the method of the present invention comprises the following steps: (1) the generation of a list of genes with significant differential expression; (2) the identification of cis-regulatory regions within the differentially expressed genes; (3) the mapping of transcription factor binding sites on the cis-regulatory regions identified; and (4) the statistic analysis of the identified TF binding profiles.

(1) The Generation of the List of Genes with Significant Differential Expression.

The gene expression data can be retrieved from various gene expression related databases. These databases are not limited to those generated by microarray techniques. They can also include gene expression data obtained by real-time quantitative PCR, Northern blot hybridization, and other gene expression related methods, including proteomics. Exemplary databases of gene expression data are listed in Table 1 above. In addition to these already available data sets, the differentially expressed gene list can also be generated by any project-oriented specific experiments, using any of the techniques discussed above, or otherwise known in the art. According to the invention, the data retrieved from such databases, or from any other source, are intensively analyzed, especially when the data involve a large number of genes or gene sets (e.g., such as SAM analysis). A list of genes showing significant differential expression is generated, and assigned the respective gene identifiers, based on the international nomenclature committee and other genome databases, using self generated scripts. As noted before, differential gene expression is considered to be “significant” when there is at least an about two-fold, preferably at least about four-fold, more preferably at least about six-fold, most preferably at least about ten-fold difference between the expression of a given gene in a test and a reference sample, such as in normal and diseased subjects, or in various stages of disease development in a diseased subject.

(2) The Identification of Cis-Regulatory Regions of Differentially Expressed Genes.

Based on the gene list generated in (1), the full-length sequences of these genes are retrieved from various full-length gene databases (such as NCBI based refSeq, NIH based MGC consortium, Japan DBTSS, and the like) (Pruitt et al., 2001, Strausberg et al., 1999, Strausberg R L et al., 2002, Yamashita et al., 2001). These full-length sequences are then compared with most updated human genome sequence databases (Lander et al., 2001, McPherson et al., 2001) (such as Human Genome Working Draft, build 31, November 2002) for mapping their chromosomal location using, for example, the BLAT software (Kent, 2002). Depending on the particular purpose, the cis-regulatory region, such as, for example, the 5′ upstream core promoter region, the 5′ upstream enhancer region, intron region, and/or 3′ regulatory region, is defined and the corresponding genomic sequences are retrieved from the most up-dated genome sequence databases (UCSC genome browser) (Kent et al., 2002, Karolchik et al., 2003). If necessary, the sequence-retrieving process can be facilitated by using self-developed scripts.

(3) Mapping of Regulatory Factor Binding Profiles on the Cis-Regulatory Regions Identified.

The genomic sequences for regulatory regions identified are screened for any putative regulatory factor binding sites, such as TF binding sites. For instance, the core promoter regions of the differentially expressed genes can be analyzed using known transcription factor binding sites. Software available for this kind of analysis is disclosed, for example, in the following publications: Grabe, 2002, Kel-Margoulis et al., 2000, Kel et al., 1995, Liebich et al., 2002, Perier et al., 2000, Praz et al., 2002, Prestridge, 1996, Quandt et al., 1995, Tsunoda et al., 1999, and Wingender, 1994. These genomic sequences of regulatory regions can be further screened for putative cis-regulatory binding sites using various motif-finding software. This can be instrumental in mapping unknown transcription factor binding sites unknown regulatory factor consensus motifs.

(4) Statistic Analysis of the Regulatory Factor Binding Profiles.

The putative regulatory factor binding sites identified in the differentially expressed genes are compared with their genome-wide or tissue-wide occurrence. The number of such binding sites, the frequencies of such binding profiles and the distribution and frequencies of occurrence are calculated, using statistical analysis. Statistical analysis can be performed, for example, using the hypergeometric distribution models, which determine the total number of successes in a fixed size sample drawn without replacement from a finite population. In particular, the hypergeometric distribution analysis (by using Microsoft Excel building function in combination with self-developed script) can be used to test if the appearances of certain regulatory factor (e.g. TF) binding sites are significantly enriched in the differential expression gene list. Such enrichment may result in abnormalities, such as tumor, e.g. cancer, when comparing with the genomic or tissue background. If necessary, the regulatory factor, e.g. TF can be identified and its sequence provided, based upon such statistical analysis. Such regulatory factors, e.g. TFs are valuable targets for therapeutic intervention directed to the prevention or treatment of diseases, disorders, or unwanted biological processes.

It will be apparent to those skilled in the art that other statistical methods can also be employed, as long as they are suitable for the comparison of frequencies or probabilities of the occurrences of regulatory regions in the genes identified in any two gene sets.

In a particular embodiment, the cis-regulatory regions, e.g. regulatory factor binding sites, of differentially expressed genes are identified by the method disclosed in co-pending application Ser. No. ______, filed ______ on (Attorney docket No.: 39753-0001). In brief, according to this approach, genomic sequences of gene regulatory regions are retrieved, from public and/or proprietary databases, DNA sequence information for each retrieved gene regulatory region is screened to identify putative regulatory factor binding sites, the putative regulatory factor binding sites are profiled, and probability mapping is applied to the profiled binding sites. The probability mapping involves the identification of specific regulatory factor binding sites, such as all the putative E2F-1 transcription factor binding sites, in the regulatory regions of all genes in a gene set, e.g. a set of differentially expressed genes in a particular disease, disease state, abnormality, and the like. The probability mapping tells how many of the differentially expressed genes are likely to be transcription-regulated by a specific regulatory factor. It also indicates how much genome-wide, cell-wide, or tissue-wide, effect a specific regulator factor is expected to have.

For each binding site identified, a conservation score can be created. The conservation score is selected to cover regions where the regulatory factor (e.g. TF) binding sites are identified as well as any other measurements that indicate conservation levels between the two species including but not limited to mouse and human. A binding site with higher conservation score or the corresponding gene with higher expression level could play a more significant role than those with lower scores.

The data generated can be collected and organized in a data bank, which can facilitate the use of the information in research and drug development efforts.

It is emphasized, however, that it is not necessary to use this proprietary approach to practice the present invention. Databases that including mapping information of gene regulatory regions can be developed in many different ways. Accordingly, the present invention is by no means limited by the way of mapping and analyzing the regulatory factor binding sites of differentially expressed genes.

Examples of regulatory factor binding sites that can be identified in accordance with the present invention include, but are not limited to, the binding site for transcription factor NF-κB (AGGGGACTTTCCCA ; SEQ ID NO: 1), and for E2F-1 (TTTGGCGG; SEQ ID NO: 2).

If the initial information is a proteomic profile (e.g. a mass spectrum) showing differential protein expression levels, the corresponding genes are located and identified, and the list of genes and their corresponding protein expression levels are used in the subsequent analysis.

C. Therapeutic Identification and Transcription Factor Decoy Design

In one specific application, the statistical analysis of regulatory binding sites performed in accordance with the present invention provides a facile way for identifying targets for therapeutic drug design, and for developing various therapeutic approaches directed to the targets identified, including, but not limited to, the design of oligonucleotide decoys.

It is well possible that all diseases, including human diseases, are somehow associated with the gene transcription process. It is well known that germline mutations in genes encoding transcription factors result in malformation syndromes affecting the development of multiple body structures. Somatic mutations in genes encoding transcription factors have been shown to contribute tumorigenesis. In addition, prenatal development and postnatal physiology demonstrate that a single transcription factor can control the proliferation of progenitor cells during development, and the expression within the differentiated cells of genes products that participate in specific physiological responses. By way of example, well-studied transcription factors, such as p53, and the Smad and STAT proteins are known to play a major role in many cancers. Transcription factors have also been identified as being involved in various neuronal, cardiovascular, renal and infectious diseases, diseases of bone development, digestive diseases, diseases associated with abnormal skeletal development, and the like. For further details see, for example, Gregg L. Semenza, Transcription Factors and Human Disease, Oxford Press 1998.

Although the transcription factor protein-DNA interaction is sequence-specific, the binding site for one given transcription factor may vary by several base pairs within different target genes. The common part, or non-variable part, of the binding sequence for a particular transcription factor is referred to as the transcription factor consensus sequence. For example, the consensus sequence for transcription factor NF-κB is AGGGGACTTTCCCA (SEQ ID NO: 1); and for E2F-1 is TTTGGCGG (SEQ ID NO: 2). The AP-1 transcription factor binds to the TGACTCA (SEQ ID NO: 3) consensus sequence. The consensus sequence for the Smad-3 transcription factor, which mediates TGF-β, activin and BMP-induced changes in gene expression is TGTCTGTCT (SEQ ID NO: 4).

If any of such consensus sequences are enriched in a biological sample representing a disease, disorder or pathologic condition, the corresponding transcription factor is a promising target of novel therapeutic approaches directed to such disease, disorder or condition.

According to the transcription factor decoy approach, small double-stranded oligonucleotides are introduced into cells to specifically bind to target transcription factors, thereby, preventing these factors from transactivating (i.e. “turning on”) their target genes.

In preclinical studies, pressure mediated ex vivo delivery of E2F Decoy has shown to prevent both neointimal hyperplasia and atherosclerosis in vein grafts of an animal model of vein graft transplantation. For more information, see, e.g. Ehsan, A., M. J. Mann 2001; Mann and Dzau 2000; Mann et al. 1999; and U.S. Pat. Nos. 5,766,901 and 5,992,687.

Further details of the invention are illustrated by the following non-limiting examples.

### EXAMPLE 1

The method of the invention was applied to a set of cell cycle related gene expression data (Whitfield et al., 2002). Proper regulation of the cell division cycle is crucial to the growth and development of all organisms; understanding this regulation is central to the study of many diseases, most notably cancer.

The genome-wide program of gene expression during the cell division cycle in a human cancer cell line (HeLa) was characterized using cDNA microarrays. Transcripts of more than 850 genes showed periodic variation during the cell cycle. Hierarchical clustering of the expression patterns revealed coexpressed groups of previously well-characterized genes involved in essential cell cycle processes such as DNA replication, chromosome segregation, and cell adhesion along with genes of uncharacterized function. Most of the genes whose expression had previously been reported to correlate with the proliferative state of tumors were found to be periodically expressed during the HeLa cell cycle. The data in this report provide a comprehensive catalog of cell cycle regulated genes that can serve as a starting point for the method of the present invention. The full dataset was retrieved from http://genome-www.stanford.edu/Human-CellCycle/HeLa/ site for further analysis.

In order to identify the key elements involved in above differential expressed genes in cell cycles, the full-length sequences of these genes were retrieved, using the combination of UCSC genome browser (Karolchik et al., 2003, Kent et al., 2002), MGC gene collection database and DBTSS databases. The transcription start site positions were mapped to the newest human genome working draft (McPherson et al, 2001, Lander et al., 2001) using the BLAT program. The sequences for core promoter regions (which is about 250 bp upstream and 50 bp downstream to the transcription start site, respectively) were retrieved using self-generated perl script for all the genes. The analysis of putative TF binding profile was performed using the Match program (Matys et al., 2003) embedded inside the licensed TRANSFAC database, combined with self-generated perl scripts.

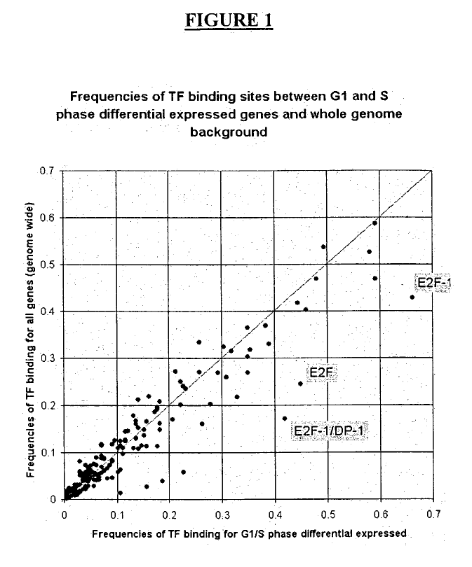

The initial screenings were performed using well-studied known transcription factors identified only from mammalian species. A typical cell cycle is composed of G1, G2, M and S phases. Ammong them, the G2 and M phases are very short relative to the G1 and S phases, which suggests that the cell phases at G1 and S are easier to define. Therefore, the focus of the present analysis has been on those differentially expressed genes (total 198) that were found in the G1 and S phases. The frequencies of the known TF binding sites identified from the above analysis were scatter-plotted against their corresponding frequencies in the genome background. The results are shown in FIG. 1. The plotting suggests that if the TF binding sites identified are normally distributed in the target gene list, the corresponding spots should locate around the red line (which is the theoretic-value if the identified TF binding frequency is the same as the corresponding genomic frequency). However, if the enrichments of certain TF-bindings indeed exist in the differentially expressed genes, the corresponding spots will be shifted away from the theoretic red line, and be moved toward the x-axis that represents the frequencies of TF-bindings in the targeted gene list. As shown in FIG. 1, the 3 most shifted spots in the target gene list, which show higher appearances (higher frequencies, >0.4) belong to the transcription factors E2F-1, E2F-1/DP-1, and E2F.

The results were subjected to further statistics analysis. The 14 TFs with highest frequencies identified in the target gene list are listed in the following Table 3, together with their P values (the right tail cumulated) of Hypergeometric Distribution test (see table). The data set forth in Table 3 suggest that E2F-1, Elk-1, E2F, and E2F-1/DP-1 are the most significant ones with the smallest P value. Like E2F-1, transcription factor Elk-1 has also been intensively studied and shown the important role in cell cycles and proliferations.

TABLE 3

| | | | P of |
|---|---|---|---|
| | Freq. of TF binding | Freq. of genomic | Hypergeometric |
| Name of TF | in target gene list | TF binding | Distribution |
| E2F-1 | 0.661616162 | 0.428784151 | 0.00000008* |
| Elk-1 | 0.590909091 | 0.469247702 | 0.0003617* |
| Pax-4 | 0.590909091 | 0.586430144 | 0.47923023 |
| MAZ | 0.580808081 | 0.525767189 | 0.06789041 |
| TFII-I | 0.494949495 | 0.536514308 | 0.89462549 |
| HNF-4 | 0.47979798 | 0.468470802 | 0.40087184 |
| c-Myc/Max | 0.45959596 | 0.402563771 | 0.05840235 |
| E2F | 0.449494949 | 0.244853036 | 0.0000001* |
| Xvent-1 | 0.444444444 | 0.417713324 | 0.24291237 |
| E2F-1/DP-1 | 0.419191919 | 0.171112262 | 0.00000001* |
| c-Ets-1(p54) | 0.388888889 | 0.330182572 | 0.04665969 |
| Sp3 | 0.383838384 | 0.369092322 | 0.35791823 |
| TCF-1(P) | 0.353535354 | 0.318205361 | 0.15923196 |
| c-Rel | 0.348484848 | 0.302214165 | 0.08983233 |

In conclusion, the key transcription factors E2F-1 and Elk-1 have been identified as factors that may play the essential role affecting 850 genes with differential expression found during the specific cell cycles processes. The cell cycles have been shown crucial in many different kinds of tumor or cancer developments. The immediate benefit from this is that one can develop therapeutic strategies based on these key elements. The transcription factor decoy (e.g., for E2F-1 Decoy, Corgentech Inc.) or anti-sense oligonucleotides are the examples for such novel treatment options. The role of E2F-1 and Elk-1 in cell proliferations was gradually developed after numerous experiments and years studies. However, our invention make this time-consuming process an easy and fast task.

All references cited throughout the disclosure, and all references cited therein are hereby expressly incorporated by reference in their entirety.

One skilled in the art will recognize many methods and materials similar or equivalent to those described herein, which could be used in the practice of the present invention. Indeed, the present invention is in no way limited to the methods and materials described.

References

Ehsan, A., M. J. Mann, G. Dell'Acqua, and V. J. Dzau. (2001). Long-term stabilization of vein graft wall architecture and prolonged resistance to experimental atherosclerosis after E2F decoy oligonucleotide gene therapy. Journal of Thoracic Cardiovascular Surgery, 121,714-722.

Grabe N. AliBaba2: context specific identification of transcription factor binding sites. In Silico Biol. 2002;2(1):S1-15.

Heinemeyer T, Chen X, Karas H, Kel A E, Kel O V, Liebich I, Meinhardt T, Reuter I, Schacherer F, Wingender E. Expanding the TRANSFAC database towards an expert system of regulatory molecular mechanisms. Nucleic Acids Res. 1999 Jan. 1;27(1):318-22.

Heinemeyer T, Wingender E, Reuter I, Hermjakob H, Kel A E, Kel O V, Ignatieva E V, Ananko E A, Podkolodnaya O A, Kolpakov F A, Podkolodny N L, Kolchanov N A. Databases on transcriptional regulation: TRANSFAC, TRRD and COMPEL. Nucleic Acids Res. 1998 Jan. 1;26(1):362-7.

Karas H, Kel' E, Kel' O V, Kolchanov N A, Wingender E. [Integrating knowledge on transcriptional regulation of eukaryotic genes based on information from TRANSFAC, TRRD, and COMPEL databases] Mol Biol (Mosk). 1997 July-August;31(4):637-46.

Kel-Margoulis O V, Romashchenko A G, Kolchanov N A, Wingender E, Kel A E. COMPEL: a database on composite regulatory elements providing combinatorial transcriptional regulation. Nucleic Acids Res. 2000 Jan. 1;28(1):311-5.

Knuppel R, Dietze P, Lehnberg W, Frech K, Wingender E. TRANSFAC retrieval program: a network model database of eukaryotic transcription regulating sequences and proteins. J Comput Biol. 1994 Fall;1(3):191-8.

Karolchik D, Baertsch R, Diekhans M, Furey T S, Hinrichs A, Lu Y T, Roskin K M, Schwartz M, Sugnet C W, Thomas D J, Weber R J, Haussler D, Kent W J. The UCSC Genome Browser Database. Nucleic Acids Res. 2003 Jan. 1;31(1):51-4.

Kent W J, Sugnet C W, Furey T S, Roskin K M, Pringle T H, Zahler A M, Haussler D. The human genome browser at UCSC. Genome Res. 2002 June;12(6):996-1006.

Kent W J. BLAT—the BLAST-like alignment tool. Genome Res. 2002 April; 12(4):656-64.

Kel A E, Kondrakhin Y V, Kolpakov Ph A, Kel O V, Romashenko A G, Wingender E, Milanesi L, Kolchanov N A. Computer tool FUNSITE for analysis of eukaryotic regulatory genomic sequences. Proc Int Conf Intell Syst Mol Biol. 1995;3:197-205.

Krull M, Voss N, Choi C, Pistor S, Potapov A, Wingender E. TRANSPATH((R)): an integrated database on signal transduction and a tool for array analysis. Nucleic Acids Res. 2003 Jan. 1;31(1):97-100.

Lander et al., 2001. Initial sequencing and analysis of the human genome. Nature. 2001 Feb. 15;409(6822):860-921.

Levy S, Hannenhalli S. Identification of transcription factor binding sites in the human genome sequence. Mamm Genome. 2002 September;13(9):510-4.

Liebich I, Bode J, Frisch M, Wingender E. S/MARt DB: a database on scaffold/matrix attached regions. Nucleic Acids Res. 2002 Jan. 1 ;30(1):372-4.

Mann, M. J., A. D. Whittemore, M. C. Donaldson, M. Belkin, M. S. Conte,J. F. Polak, E. J. Orav, A. Ehsan, G. Dell'Acqua, and V. J. Dzau. (1999). Ex-vivo gene therapy of human vascular bypass grafts with E2F decoy: the PREVENT single-centre, randomised, controlled trial. Lancet, 354, 1493-1498.

Mann, M. J., and V. J. Dzau. (2000). Therapeutic applications of transcription factor decoy oligonucleotides. Journal of Clinical Investigation, 106, 1071-1075.

Matys V, et al. TRANSFAC: transcriptional regulation, from patterns to profiles. Nucleic Acids Res. 2003 Jan. 1;31(1):374-8.

McPherson et al, 2001. A physical map of the human genome. Nature. 2001 Feb. 15;409(6822):934-41.

Perier R C, Praz V, Junier T, Bonnard C, Bucher P. The eukaryotic promoter database (EPD). Nucleic Acids Res. 2000 Jan. 1;28(1):302-3.

Praz V, Perier R, Bonnard C, Bucher P. The Eukaryotic Promoter Database, EPD: new entry types and links to gene expression data. Nucleic Acids Res. 2002 Jan. 1 ;30(1):322-4.

Prestridge D S. SIGNAL SCAN 4.0: additional databases and sequence formats. Comput Appl Biosci. 1996 April;12(2):157-60.

Pruitt K D, Maglott D R. RefSeq and LocusLink: NCBI gene-centered resources. Nucleic Acids Res. 2001 Jan. 1;29(1):137-40.

Quandt K, Frech K, Karas H, Wingender E, Werner T. Matlnd and MatInspector: new fast and versatile tools for detection of consensus matches in nucleotide sequence data. Nucleic Acids Res. 1995 Dec. 11;23(23):4878-84.

Schacherer F, Choi C, Gotze U, Krull M, Pistor S, Wingender E. The TRANSPATH signal transduction database: a knowledge base on signal transduction networks. Bioinformatics. 2001 November; 17(11): 1053-7.

Strausberg R L, Feingold E A, Klausner R D, Collins F S. The mammalian gene collection. Science. 1999 Oct. 15;286(5439):455-7.

Strausberg R L et al. Generation and initial analysis of more than 15,000 full-length human and mouse cDNA sequences. Proc Natl Acad Sci USA. 2002 Dec. 24;99(26): 16899-903.

Tsunoda T, Takagi T. Estimating transcription factor bindability on DNA. Bioinformatics. 1999 July-August;15(7-8):622-30.

Whitfield M L, Sherlock G, Saldanha A J, Murray J I, Ball C A, Alexander K E, Matese J C, Perou C M, Hurt M M, Brown P O, Botstein D. Identification of genes periodically expressed in the human cell cycle and their expression in tumors. Mol Biol Cell. 2002 June; 13(6): 1977-2000.

Wingender E, Chen X, Fricke E, Geffers R, Hehl R, Liebich I, Krull M, Matys V, Michael H, Ohnhauser R, Pruss M, Schacherer F, Thiele S, Urbach S. The TRANSFAC system on gene expression regulation. Nucleic Acids Res. 2001 Jan. 1;29(1):281-3.

Wingender E, Chen X, Hehl R, Karas H, Liebich I, Matys V, Meinhardt T, Pruss M, Reuter I, Schacherer F. TRANSFAC: an integrated system for gene expression regulation. Nucleic Acids Res. 2000 Jan. 1;28(1):316-9.

Wingender E, Karas H, Knuppel R. TRANSFAC database as a bridge between sequence data libraries and biological function. Pac Symp Biocomput. 1997;:477-85.

Wingender E, Kel A E, Kel O V, Karas H, Heinemeyer T, Dietze P, Knuppel R, Romaschenko A G, Kolchanov N A. TRANSFAC, TRRD and COMPEL: towards a federated database system on transcriptional regulation. Nucleic Acids Res. 1997 Jan. 1;25(1):265-8.

Wingender E, Dietze P, Karas H, Knuppel R. TRANSFAC: a database on transcription factors and their DNA binding sites. Nucleic Acids Res. 1996 Jan. 1;24(1):238-41.

Wingender E. Recognition of regulatory regions in genomic sequences. J Biotechnol. 1994 Jun. 30;35(2-3):273-80.

Suzuki Y, Yamashita R, Nakai K, Sugano S. DBTSS: DataBase of human Transcriptional Start Sites and full-length cDNAs.

What is claimed is:

1. A method for statistical analysis of differentially expressed genes, comprising:
(a) obtaining a set of differentially expressed genes;
(b) screening genomic sequences including the regulatory regions of said differentially expressed genes for the presence of regulatory factor binding sites; and
(c) identifying at least one regulatory factor binding site enriched within said set of differentially expressed genes relative to a genome-wide or tissue-wide background.

2. The method of claim 1 wherein in step (c) enrichment is determined by comparing the frequency or probability of the occurrence of the regulatory binding site or binding sites identified in step (c) within said gene set with the frequency or probability of their occurrence in a genome-wide or tissue-wide background.

3. The method of claim 1 wherein prior to obtaining said set of differentially expressed genes, a proteomic profile of a set of differentially expressed proteins is obtained.

4. The method of claim 1 wherein said set of differentially expressed genes is part of a gene expression profile characteristic of a disease, disorder, or biological process.

5. The method of claim 4 wherein said disease is selected from the group consisting of tumor, oncological diseases, neurological diseases, cardiovascular diseases, renal diseases, infectious diseases, digestive diseases, metabolic diseases, inflammatory diseases, autoimmune diseases, dermatological diseases, and diseases associated with trauma or abnormal skeletal development.

6. The method of claim 5 wherein said tumor is cancer.

7. The method of claim 6 wherein said cancer is selected from the group consisting of breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, and brain cancer.

8. The method of claim 4 wherein said disorder is a developmental disorder.

9. The method of claim 4 wherein said biological process is associated with aging.

10. The method of claim 1 wherein said set consists of genes that show at least about two-fold differential expression relative to control.

11. The method of claim 1 wherein said set consists of genes that show at least about four-fold differential expression relative to control.

12. The method of claim 1 wherein said set consists of genes that show at least about ten-fold differential expression relative to control

13. The method of claim 1 wherein said regulatory factor binding site is identified within a region selected from the group consisting of a 5′ upstream core promoter region, a 5′ upstream enhancer region, an intron region, and a 3′ regulatory region.

14. The method of claim 13 wherein said regulatory factor binding site is a transcription factor binding site.

15. The method of claim 14 wherein said transcription factor is selected from the group consisting of c-Fos, c-Jun, AP-1, Elk, ATF, c-Ets-1, c-Rel, CRF, CTF, GATA-1, POU1F1, NF-κB, POU2F1, POU2F2, p53, Pax-3, Sp1, TCF, TAR, TFEB, TCF-1, TFIIF, E2F-1, E2F-2, E2F-3, E2F-4, HIF-1, HIF-1α, HOXA1, HOXA5, Sp3, Sp4, TCF-4, APC, and STAT5A.

16. The method of claim 15 wherein said transcription factor is selected from the group consisting of E2F-1, E2F-2, E2F-3, NF-κB, Elk, AP-1, c-Fos, and c-Jun.

17. The method of claim 1 wherein at least 50 differentially expressed genes are analyzed.

18. The method of claim 1 wherein at least 100 differentially expressed genes are analyzed.

19. The method of claim 1 wherein at least 500 differentially expressed genes are analyzed.

20. The method of claim 1 further comprising the step of designing a treatment strategy based upon the identification of said enriched regulatory factor binding site.

21. The method of claim 20 wherein said enriched regulatory factor binding site is a transcription factor binding site binding to at least one transcription factor.

22. The method of claim 21 wherein a consensus binding site is identified based on said enriched transcription factor binding site.

23. The method of claim 20 wherein said treatment strategy relies on the design of a double-stranded oligonucleotide decoy, which competes with said enriched binding site for binding to the corresponding transcription factor.

24. The method of claim 20 wherein said treatment strategy relies on an anti-sense oligonucleotide designed to bind to said enriched binding site.

25. A method of designing a consensus regulatory factor binding site, comprising identifying a regulatory factor binding site enriched within a set of differentially expressed genes, relative to a genome-wide or tissue-wide control, and designing a consensus regulatory factor binding site consisting essentially of nucleotides shared by the regulatory factor binding sites enriched within said set of differentially expressed genes.

26. A method of analyzing the enrichment of a regulatory factor binding site in a biological sample comprising a set of differentially expressed genes, comprising comparing the frequency or probability of the occurrence of said regulatory binding site within said gene set with the frequency or probability of its occurrence in a reference sample.

27. The method of claim 26 wherein the biological sample is a tissue sample.

28. The method of claim 27 wherein the tissue comprises tumor cells.

29. The method of claim 28 wherein the tissue comprises cancer cells.

30. The method of claim 28 wherein the cancer is selected from the group consisting of breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, and brain cancer.

31. The method of claim 28 wherein the reference sample is a normal tissue of the same tissue type.

32. The method of claim 28 wherein the reference sample is the human genome.

33. The method of claim 26 wherein the biological sample is a biological fluid.

34. The method of claim 26 wherein the enrichment is determined by using hypergeometric distribution analysis.


SEQUENCE LISTING (4 sequences)

SEQ ID NO: 1 - DNA, 14 bp, Homo Sapiens
aggggactttccca

SEQ ID NO: 2 - DNA, 8 bp, Homo Sapiens
tttggcgg

SEQ ID NO: 3 - DNA, 7 bp, Homo Sapiens
tgactca

SEQ ID NO: 4 - DNA, 9 bp, Homo Sapiens
tgtctgtct